(19) United States
(12) Patent Application Publication
(10) Pub. No.: US 2018/0185093 A1
(43) Pub. Date: Jul. 5, 2018

(54) URETEROSCOPE AND A METHOD FOR DUSTING STONES IN A BODY CAVITY WITH A LASER FIBER

(71) Applicant: Wipro Limited, Bangalore (IN)

(72) Inventors: Pavan MISRA, Bangalore (IN); Gunalan SELVAM, Bangalore (IN); Brijesh BABU, Bangalore (IN); Sandesh GAVADE, Bangalore (IN)

(73) Assignee: Wipro Limited

(21) Appl. No.: 15/430,787

(22) Filed: Feb. 13, 2017

(30) Foreign Application Priority Data
Dec. 30, 2016 (IN) 201641044978

(51) Int. Cl. A61B 18/24; A61B 1/018; A61B 1/307; A61B 1/00

(52) U.S. Cl. CPC A61B 18/245; A61B 1/018; A61B 1/307; A61B 2018/00511; A61B 1/00098; A61B 1/00133; A61B 1/00006; A61B 1/00066

(57) ABSTRACT

The present disclosure relates to a ureteroscope and a method for dusting stones in a body cavity with a laser fiber. The ureteroscope includes a flexible probe and a handle. The flexible probe includes a working channel that accommodates the laser fiber in a distal end of the flexible probe at a working position to be deflected against a target stone for dusting. The handle includes a plurality of control switches and a laser fiber feeder and retraction module. The control switches are configured to provide digital control signals to control a motor assisted movement of the laser fiber in the working channel. The motor assisted movement of the laser fiber includes one of a feeding movement and a retracting movement. The laser fiber feeder and retraction module is configured to control one of the feeding movement and the retracting movement of the laser fiber in the working channel.

### FIELD OF THE DISCLOSURE

The present subject matter generally relates to field of ureteroscopes. More particularly, the present disclosure discloses a ureteroscope and a method for dusting stones in a body cavity with a laser fiber.

### BACKGROUND

Ureteroscopy is a method used in treatment of ureteral stones and kidney stones using ureteroscopes. The ureteroscopes have reduced in size and become technologically advanced to facilitate ureteroscopic management of the ureteral stones and the kidney stones.

Currently, an urologist is required to manually feed or retract a laser fiber into a ureteroscope after inserting the ureteroscope into a natural path of a patient to target a stone either in a ureter or a kidney. An assistant is required to hold the ureteroscope during feeding or retracting of the laser fiber by the urologist. In case the assistant moves the ureteroscope, the urologist loses sight of the stone and will again have to pass the laser fiber to somebody else to hold, maneuver the ureteroscope to get a clear vision of the stone and then restart the ureteroscopy using the laser fiber as mentioned above. The urologist further may perform a method of physically twitching and tweaking a laser fiber to break the stone. Such a method is also referred to as dusting. The dusting refers to a process in which a deflecting tip of the ureteroscope having the laser fiber is swayed from one side to another and a low energy high frequency laser pulse is used to break down the stone into fine dust without breaking the stone into big fragments. The fine dust is washed away, by saline flowing in through the ureteroscope, out of a urinary system of the patient through the natural path. Such a method of dusting with the laser fiber can be a tedious task and also adds to a load on the urologist and the patient due to duration of such dusting. Such a procedure of the dusting also has increased probability of damaging internal tissues of the urinary system if performed incorrectly or due to less control on deflection of the deflecting tip. Moreover, longer procedure time can also affect post-op recovery times and clinical outcome of the procedure.

With the urologist manually managing the laser fiber by physically twitching and tweaking the laser fiber, there are chances of breakage of the laser fiber which is brittle due to inconsistent force being applied on the laser fiber. If the laser fiber breaks, the urologist has to remove the laser fiber from the ureteroscope and insert another laser fiber, which may happen repeatedly. Such repeated replacements of the laser fiber leads to a financial loss for the urologist as laser fibers are expensive.

### SUMMARY

Embodiments of present disclosure disclose an ureteroscope for dusting stones in a body cavity with a laser fiber. The ureteroscope includes a flexible probe and a handle. The handle extends proximally from the flexible probe. The flexible probe includes a proximal end and a distal end. The flexible probe includes a working channel that is configured to accommodate a laser fiber in the distal end of the flexible probe at a working position. The laser fiber is deflected against a target stone for dusting. The handle includes a plurality of control switches and a laser fiber feeder and retraction module. The plurality of control switches is configured to provide digital control signals. The digital control signals are configured to control a motor assisted movement of the laser fiber in the working channel by a user. The motor assisted movement of the laser fiber includes one of a feeding movement and a retracting movement. The laser fiber feeder and retraction module is configured to control one of the feeding movement and the retracting movement of the laser fiber in the working channel. The feeding movement of the laser fiber includes a forward movement of the laser fiber in a first rotating direction and the retracting movement of the laser fiber includes a backward movement of the laser fiber in a second rotating direction.

Disclosed herein is a method of dusting stones in a body cavity with a laser fiber in a ureteroscope. The method includes receiving, by a processing module of a laser fiber feeder and retraction module of the ureteroscope, a digital light dependent resistor signal from a light dependent resistor (LDR) and digital control signals from a plurality of control switches. The digital LDR signal is generated in response to sensing presence of a laser fiber in a working channel of a flexible probe of the ureteroscope. The laser fiber is manually introduced into the working channel and positioned at an initial position and configured to be advanced to a working position. The digital control signals are configured to control a motor assisted movement of the laser fiber in the working channel by a user. The motor assisted movement of the laser fiber includes one of a feeding movement and a retracting movement. The method also includes processing, by the processing module, the digital LDR signal and the digital control signals to generate an analog motor signal. Further, the method includes initiating, by the processing module, activation of a motor in the laser fiber feeding and retraction module in response to the analog motor signal. The motor controls one of the feeding movement and the retracting movement of the laser fiber in the working channel for dusting of a target stone. The feeding movement of the laser fiber includes a forward movement of the laser fiber in a first rotating direction, and the retracting movement of the laser fiber includes a backward movement of the laser fiber in a second rotating direction.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

The accompanying drawings, which are incorporated in and constitute a part of this disclosure, illustrate exemplary embodiments and, together with the description, serve to explain the disclosed principles. In the figures, the left-most digit(s) of a reference number identifies the figure in which the reference number first appears. The same numbers are used throughout the figures to reference like features and components. Some embodiments of system and/or methods in accordance with embodiments of the present subject matter are now described, by way of example only, and with reference to the accompanying figures, in which:

FIG. 1 illustrates a side view of a ureteroscope for dusting stones in a body cavity with a laser fiber, in accordance with some embodiments of the present disclosure;

FIG. 2 illustrates a cross-sectional view of a deflecting tip of a ureteroscope, in accordance with some embodiments of the present disclosure;

FIG. 3 illustrates a side view of a ureteroscope for dusting stones in a body cavity with a laser fiber, in accordance with other embodiments of the present disclosure;

FIG. 4 illustrates a block diagram of a processing module of a laser fiber feeder and retraction module in a ureteroscope, in accordance with some embodiments of the present disclosure; and

FIG. 5 is a flow diagram illustrating a method of dusting stones in a body cavity with a laser fiber in a ureteroscope, in accordance with some embodiments of the present disclosure.

It should be appreciated by those skilled in the art that any block diagrams herein represent conceptual views of illustrative systems embodying the principles of the present subject matter. Similarly, it will be appreciated that any flow charts, flow diagrams, state transition diagrams, pseudo code, and the like represent various processes which may be substantially represented in computer readable medium and executed by a computer or processor, whether or not such computer or processor is explicitly shown.

### DETAILED DESCRIPTION

In the present document, the word “exemplary” is used herein to mean “serving as an example, instance, or illustration,” Any embodiment or implementation of the present subject matter described herein as “exemplary” is not necessarily to be construed as preferred or advantageous over other embodiments.

While the disclosure is susceptible to various modifications and alternative forms, specific embodiment thereof has been shown by way of example in the drawings and will be described in detail below. It should be understood, however that it is not intended to limit the disclosure to the particular forms disclosed, but on the contrary, the disclosure is to cover all modifications, equivalents, and alternative falling within the scope of the disclosure.

The terms “comprises”, “comprising”, or any other variations thereof, are intended to cover a non-exclusive inclusion, such that a setup, device or method that comprises a list of components or steps does not include only those components or steps but may include other components or steps not expressly listed or inherent to such setup or device or method. In other words, one or more elements in a system or apparatus proceeded by “comprises . . . a” does not, without more constraints, preclude the existence of other elements or additional elements in the system or apparatus.

In the following detailed description of the embodiments of the disclosure, reference is made to the accompanying drawings that form a part hereof, and in which are shown by way of illustration specific embodiments in which the disclosure may be practiced. These embodiments are described in sufficient detail to enable those skilled in the art to practice the disclosure, and it is to be understood that other embodiments may be utilized and that changes may be made without departing from the scope of the present disclosure. The following description is, therefore, not to be taken in a limiting sense.

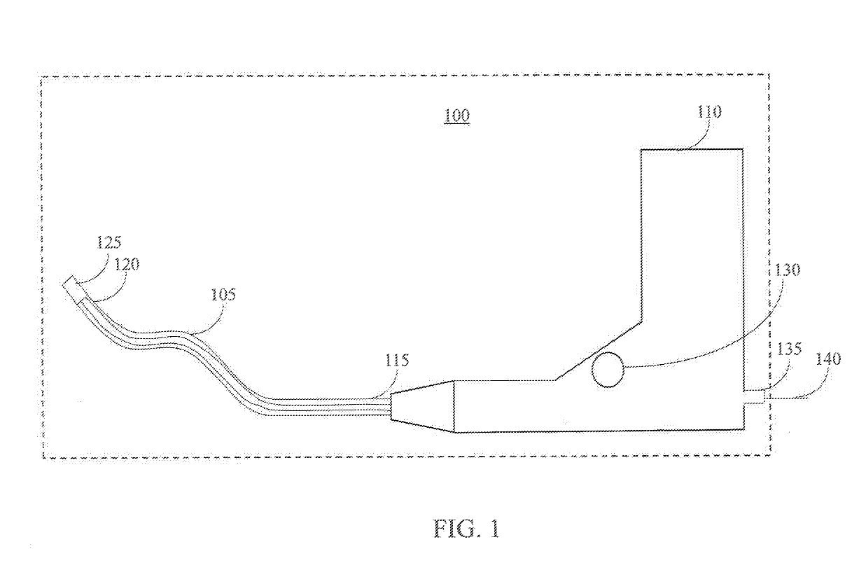

FIG. 1 illustrates a side view of a ureteroscope 100 for dusting stones in a body cavity with a laser fiber, in accordance with some embodiments of the present disclosure.

The ureteroscope 100 includes a flexible probe 105 and a handle 110. The handle 110 extends proximally from the flexible probe 105. The flexible probe 105 includes a proximal end 115 and a distal end 120. The proximal end 115 of the flexible probe 105 is coupled to the handle 110. The flexible probe 105 includes at least one working channel and a deflecting tip 125. The deflecting tip 125 of the flexible probe 105 is coupled to the distal end 120. The flexible probe 105 further includes an image viewing system (not shown in FIG. 1) and a light source (not shown in FIG. 1). The handle 110 includes a plurality of control switches, for example a control switch 130, on an exterior portion of the handle 110. The handle 110 also includes a working channel 135, on an exterior portion of the handle 110, to receive a laser fiber 140. The working channel 135 continues into the flexible probe 105. The handle 110 further includes a laser fiber feeder and retraction module (not shown in FIG. 1) in an interior portion of the handle 110.

The ureteroscope 100 can be used in a stone removal operation from a kidney of a patient. A user, for example a doctor or other medical personnel, inserts the flexible probe 105 of the ureteroscope 100 into a urethra of the patient. The user advances the flexible probe 105 such that the deflecting tip 125 passes into and through a urinary bladder, into and through a ureter, and into a kidney of the patient.

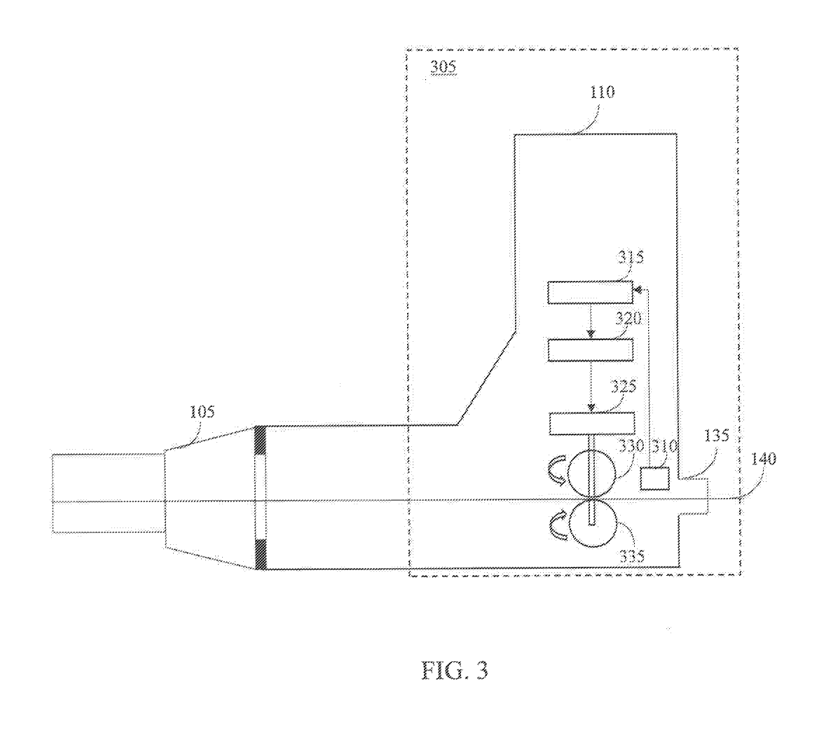

The user positions the deflecting tip 125 of the ureteroscope 100 within the kidney at a minimum distance from a target stone. Once the deflecting tip 125 of the ureteroscope 100 is positioned in the kidney, the laser fiber 140 is received into the working channel 135 of the flexible probe 105 at an initial position and advanced into the working channel 135. Herein, the ‘initial position’ is referred to as a predefined distance of the laser fiber 140 in the working channel 135 prior to a feeding movement of the laser fiber 140 by the laser fiber feeder and retraction module. The laser fiber 140 is accommodated in the distal end 120 of the flexible probe 105 at a working position. In an example the laser fiber 140 may include a diameter of 200 micrometer, 272 micrometer, 365 micrometer, 550 micrometer, or 1000 micrometer. Herein, the ‘working position’ is referred to as a predefined distance of the laser fiber 140 from the distal end 120 of the flexible probe 105 defined by laser energy settings and stone composition. The user operates the control switch 130 to control a motor assisted movement of the laser fiber 140 in the working channel 135 by the user. The motor assisted movement of the laser fiber 140 includes one of the feeding movement and a retracting movement of the laser fiber 140 in the working channel 135. The feeding movement and the retracting movement of the laser fiber 140 is described in detail with reference to FIG. 3.

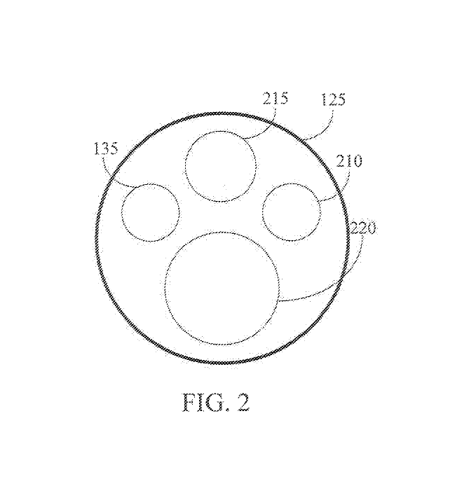

In some embodiments, the ureteroscope 100 may also be positioned in a ureter of the patient for dusting the target stone in the ureter with the laser fiber 140. The deflecting tip 125 including the working channel 135 is explained in detail with reference to FIG. 2.

Referring now to FIG. 2, a cross sectional view of the deflecting tip 125 including the working channel 135 is illustrated, in accordance with some embodiments of the present disclosure. In some embodiments, the deflecting tip 125 may be composed of a plurality of segmented portions held together with a plurality of control wires such that the deflecting tip 125 is flexible. As illustrated in FIG. 2, the deflecting tip 125 includes the working channel 135 and a working channel 210. The working channel 135 is configured to accommodate the laser fiber 140 for dusting the target stone. In some embodiments, the working channel 135 may accommodate a 3 french gauge (Fr) size working device. In other embodiments, the working channel 135 may be used to accommodate baskets, grasping forceps, and the like. The working channel 210 is configured to be used for irrigation, for example with saline, to wash away dust after the dusting of the target stone with the laser fiber 140.

The deflecting tip 125 further includes an image viewing system 215 and a light source 220. In one example, the image viewing system 215 is a complementary metal oxide semiconductor (CMOS) based image viewing system. The image viewing system 215 is configured to view the laser fiber 140 extending from the deflecting tip 125 and also the target stone. The light source 220 is configured to provide light to assist viewing of the target stone and the laser fiber 140 through the image viewing system 215.

The feeding movement and the retraction movement of the laser fiber 140 in the ureteroscope 100 and subsequent dusting using the laser fiber 140 is explained in detail with reference to FIG. 3.

FIG. 3 illustrates a side view of the ureteroscope 100 for dusting stones in the body cavity of the patient with the laser fiber 140, in accordance with other embodiments of the present disclosure. In the illustrated FIG. 3, a laser fiber feeder and retraction module 305 of the handle 110 and the dusting of the target stone with the laser fiber 140 are described herein in detail. The laser fiber feeder and retraction module 305 is configured to control one of the feeding movement and the retracting movement of the laser fiber 140 in the working channel 135. The feeding movement of the laser fiber 140 includes a forward movement of the laser fiber 140 in a first rotating direction. The retracting movement of the laser fiber 140 includes a backward movement of the laser fiber 140 in a second rotating direction.

The handle 110 includes the laser fiber feeder and retraction module 305 (an electromechanical module) in the interior portion of the handle 110. The laser fiber feeder and retraction module 305 includes a light dependent resistor (LDR) 310, a processing module 315, a driver 320, a motor 325, and a plurality of pulleys, for example a first pulley 330 and a second pulley 335.

The flexible probe 105 of the ureteroscope 100 is inserted into the body cavity of the patient and the defecting tip 125 is deflected or maneuvered to reach a position of the target stone in the kidney or the ureter of the patient. the laser fiber 140 is then inserted into the working channel 135. The feeding movement and the retracting movement of the laser fiber 140 using the laser fiber feeder and retraction module 305 is explained below.

In the feeding movement, the laser fiber 140 is manually introduced into the working channel 135 and positioned at the initial position. The LDR. 310 senses presence of the laser fiber 140 in the working channel 135. The LDR 310 then generates a digital LDR signal in response to sensing presence of the laser fiber 140 in the working channel 135. The LDR 310 triggers the processing module 315 with the digital LDR signal to activate the motor 325. The motor 325 rotates a small step in order to grip the laser fiber 140 at the initial position.

In order to feed the laser fiber 140 further into the working channel 135, the user can press the control switch 130 to generate the digital control signals. The processing module 315 coupled to the LDR 310 generates a digital motor signal in response to the digital LDR signal and the digital control signals. The driver 320 is coupled to the processing module 315 and generates an analog motor signal in response to the digital motor signal. The motor 325, for example a stepper motor, is coupled to the driver 320. The motor 325 is configured to turn a number of steps based on the analog motor signal at a preset speed. In an example, the motor 325 may be associated with a step angle of 1.8 degrees, a holding torque of 14 Newton-centimeters, and a rotor inertia of 0.012 kilogramme square centimeter. The plurality of pulleys, for example the first pulley 330 and the second pulley 335, is coupled to the motor 325. In an example, the plurality of pulleys include biocompatible elastic polyurethane over-mold on biocompatible plastic pulleys for gripping and feeding laser fibers various diameters without slipping. In an example, the plurality of pulleys is over-molded with compressible rubber grippers to grip the laser fiber during the feeding and retracting of the laser fiber in presence of saline flowing through the working channel 210 during the dusting. The compressible rubber grippers permit the user to select between different sizes of laser fibers available for the dusting as determined by energy levels required based on kidney stone composition and sizes.

The plurality of pulleys is configured to control the motor assisted movement of the laser fiber 140 in the working channel. The plurality of pulleys push the laser fiber 140 into the working channel 135 in the forward movement of the laser fiber 140 in the first rotating direction, for example a clockwise direction. The laser fiber 140 is pushed until the working position is reached or a predefined position from the deflecting tip 125 is reached. The dusting of the target stone is further performed using the laser fiber 140 at the working position.

In some embodiments, the plurality of pulleys is included in a feeder housing that is a biocompatible plastic enclosure for mounting the plurality of pulleys.

In some embodiments, another control switch may be pressed by the user in order to achieve finer steps or position the laser fiber 140 in an appropriate position during the feeding movement. The processing module 315 instructs the motor 325 accordingly.

Subsequent to the feeding movement of the laser fiber 140 and positioning of the laser fiber 140 at the working position, the laser fiber 140 is energized to a power level and a frequency level to enable the dusting of the target stone. The laser fiber 140 can then be retracted in the retracting movement explained below.

In the retracting movement, the laser fiber 140 is already present in the working channel 135 and is positioned at the working position or the predefined position from the deflecting tip 125.

In order to retract the laser fiber 140 from the working channel 135, the user can press another control switch to trigger processing module 315 to activate the motor 325. The motor 325 rotates a small step in a reverse direction. The plurality of pulleys hence retract the laser fiber 140 from the working channel 135 in the backward movement of the laser fiber 140 in the second rotating direction, for example an anti-clockwise direction. The laser fiber 140 is retracted until the initial position is reached. The laser fiber 140 may further be manually pulled out by the user.

In some embodiments, another control switch may be pressed by the user in order to achieve finer steps or position the laser fiber 140 in an appropriate position during the retracting movement. The processing module 315 instructs the motor 325 accordingly.

In some embodiments, a laser trigger is used to energize the laser fiber and is a pedal activated module which is located away from the ureteroscope 100. In other embodiments, the laser trigger may be included within the ureteroscope 100.

In some embodiments, the image viewing system 215 and the light source 220 provides aid to the user to position the laser fiber 140 near the target stone and to perform the dusting of the target stone.

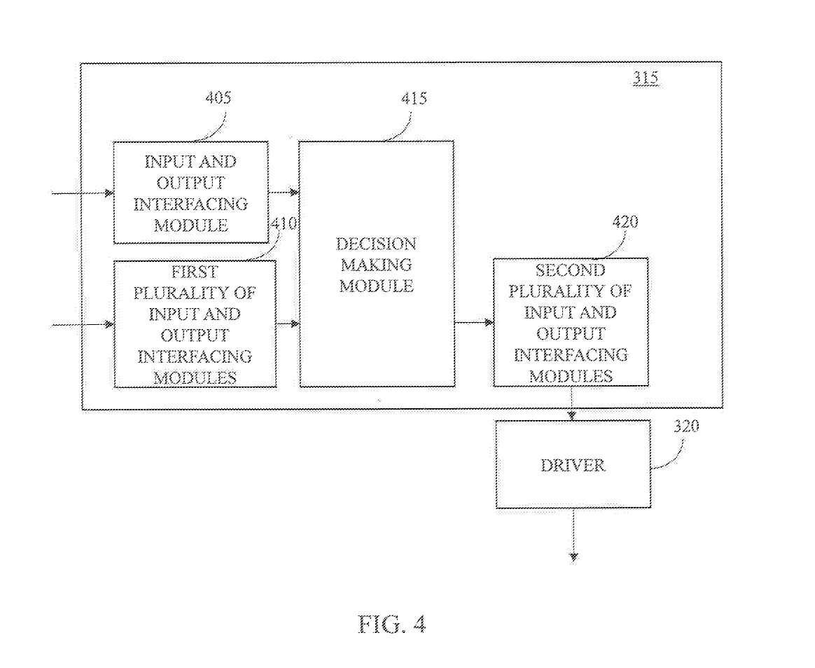

The functions of the processing module 315 during the feeding movement and the retracting movement are explained in detail with reference to FIG. 4.

Referring now to FIG. 4, the processing module 315 is illustrated, in accordance with some embodiments of the present disclosure. The processing module 315 includes an input and output interfacing module 405 and a first plurality of input and output interfacing modules 410. The processing module 315 further includes a decision making module 415. In addition, the processing module 315 includes a second plurality of input and output interfacing modules 420.

The input and output interfacing module 405 and the first plurality of input and output interfacing modules 410 are coupled to the decision making module 415. The decision making module 415 is further coupled to the second plurality of input and output interfacing modules 420. The driver 320 is coupled to the second plurality of input and output interfacing modules 420. An output of the driver 320 is further coupled to the motor 325.

Once the laser fiber 140 is fed into the working channel 135, the LDR 310 senses the presence of the laser fiber 140 and triggers the processing module 315. The input and output interfacing module 405 receives the digital LDR signal from the LDR 310. The digital LDR signal is transmitted to the decision making module 415.

The first plurality of input and output interfacing modules 410 receive the digital control signals from the plurality of control switches, for example the control switch 130. The digital control signals are received when the user selects the control switch 130 or another control switch to control the feeding movement and the retracting movement of the laser fiber 140. The digital control signals are transmitted to the decision making module 415.

The decision making module 415 is configured to determine the presence of the laser fiber 140 in the working channel 135 based on the digital LDR signal, and to determine a type of the feeding movement and the retracting movement of the laser fiber 140 based on the digital control signals. The type of the feeding movement of the laser fiber 140 includes one of a fine feed and a coarse feed of the laser fiber 140. The type of the retracting movement of the laser fiber 140 includes one of a fine retract and a coarse retract of the laser fiber 140.

The second plurality of input and output interfacing modules 420 is configured to receive an output data signal from the decision making module 415. Based on the output data signal received from the decision making module 415, the second plurality of input and output interfacing modules 420 generates the digital motor signal.

The driver 320 receives the digital motor signal from the second plurality of input and output interfacing modules 420 and in turn generates the analog motor signal. The motor 325, coupled to the driver 320, hence turns the number of steps in accordance with the analog motor signal. The motor assisted movement of the laser fiber 140 in the working channel 135 is hence controlled by controlling the plurality of pulleys coupled to the motor 325. The laser fiber 140 is energized after the feeding movement and the dusting of the target stone is performed. The retracting movement of the laser fiber 140 from the working channel 135 is subsequently performed.

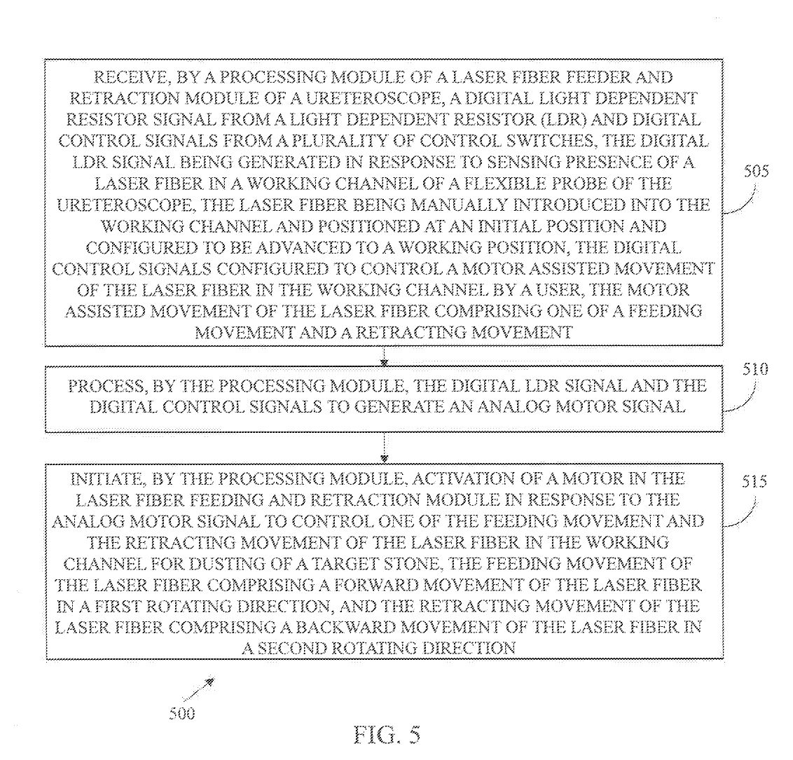

FIG. 5 is a flow diagram illustrating a method 500 of dusting stones in a body cavity with a laser fiber, for example the laser fiber 140 of FIG. 1, in a ureteroscope, for example the ureteroscope 100 of FIG. 1, in accordance with some embodiments of the present disclosure. The term ‘dusting’ refers to a technique for disintegrating stones in a kidney or a ureter of a patient into fine fragments with a laser fiber by moving a deflecting tip of the ureteroscope, for example the deflecting tip 125 of the ureteroscope 100 of FIG. 1.

The order in which the method is described is not intended to be construed as a limitation, and any number of the described method blocks can be combined in any order to implement the method. Additionally, individual blocks may be deleted from the method without departing from the scope of the subject matter described herein.

At step 505, the method 500 includes receiving a digital light dependent resistor signal from a light dependent resistor (LDR), for example the LDR 310 of FIG. 3, and digital control signals from a plurality of control switches, for example the control switch 130 of FIG. 1. The digital LDR signal and the digital control signals are received by a processing module of a laser fiber feeder and retraction module, for example the processing module 315 of the laser fiber feeder and retraction module 305, of the ureteroscope. The digital LDR signal is generated in response to sensing presence of a laser fiber in a working channel of a flexible probe of the ureteroscope, for example the laser fiber 140 in the working channel 135 of a flexible probe 105. The laser fiber is manually introduced into the working channel and positioned at an initial position and configured to be advanced to a working position.

The initial position is defined as a predefined distance of the laser fiber in the working channel prior to the feeding movement of the laser fiber by the laser fiber feeder and retraction module. The working position is defined as a predefined distance of the laser fiber from a distal end of the flexible probe and is defined by laser energy settings and composition of the target stone.

The digital control signals control a motor assisted movement of the laser fiber in the working channel by a user. The motor assisted movement of the laser fiber includes one of a feeding movement and a retracting movement of the laser fiber. At the working position, the laser fiber extends from a deflecting tip, for example the deflecting tip 125, of the flexible probe. The laser fiber is viewed by an image viewing system, for example the image viewing system 215 of FIG. 2, in the flexible probe with assistance of light provided by a light source, for example the light source 220 of FIG. 2, in the flexible probe.

At step 510, the method 500 includes processing, by the processing module, the digital LDR signal and the digital control signals to generate an analog motor signal. The analog motor signal is generated by the processing module of the laser fiber feed and retraction module. The analog motor signal is further provided to a driver of a motor, for example the driver 320 of the motor 325 of FIG. 3, in the ureteroscope. The method of generating the analog motor signal is explained with reference to FIG. 4 is not explained herein for sake of brevity.

At step 515, the method 500 includes initiating, by the processing module, activation of the motor in the laser fiber feeding and retraction module in response to the analog motor signal. The analog motor signal controls one of the feeding movement and the retracting movement of the laser fiber in the working channel for dusting of a target stone. The feeding movement of the laser fiber includes a forward movement of the laser fiber in a first rotating direction. The retracting movement of the laser fiber includes a backward movement of the laser fiber in a second rotating direction. In an example, the motor used is a stepper motor. The driving of the motor pushes forward or backward the laser fiber using a plurality of pulleys, for example the first pulley 330 and the second pulley 335 of FIG. 3. In an example, the plurality of pulleys are over-molded with compressible rubber grippers to grip the laser fiber during the feeding and retracting of the laser fiber. The method of the feeding movement and the retracting movement of the laser fiber is explained with reference to FIG. 3 is not explained herein for sake of brevity.

The feeding movement and the retracting movement of the laser fiber is controlled by one or more method steps. The method includes generating, by the processing module, an output data signal corresponding to the digital LDR signal and the digital control signals. The output data signal defines the presence of the laser fiber in the working channel based on the digital LDR signal and type of the feeding movement and the retracting movement of the laser fiber.

The method also includes processing, by the processing module, the output data signal to generate the analog motor signal.

The method further includes initiating, by the processing module, driving of the motor to turn a number of steps based on the analog motor signal to move the laser fiber in one of the feeding movement and the retracting movement of the laser fiber using a plurality of pulleys.

The laser fiber extending from the deflecting tip is deflected subsequent to the feeding movement to enable the dusting of the target stone. The laser fiber is energized to a power level and a frequency level to enable the dusting of the target stone. Subsequently, the retracting movement of the laser fiber is performed.

In some embodiments, the laser fiber is energized using a laser trigger that is a pedal activated module and which is located away from the ureteroscope. In other embodiments, the laser fiber is energized to a power level and a frequency level using the laser trigger included within the ureteroscope.

In some embodiments, the type of the feeding movement and the retracting movement of the laser fiber is determined by the processing module based on the digital control signals. The type of the feeding movement of the laser fiber includes one of a fine feed and a coarse feed of the laser fiber. The type of the retracting movement of the laser fiber includes one of a fine retract and a coarse retract of the laser fiber.

In some embodiments, after the dusting of the target stone with the laser fiber, dust of the target stone is flushed outside the body using the ureteroscope without affecting adjacent body tissues.

Embodiments of the present disclosure provide a ureteroscope and a method for dusting stones in a body cavity with a laser fiber. The present disclosure provides a controlled and motorized constant rate of feeding or retracting of a laser fiber. Such a method leads to a reduced risk of laser fiber buckling or kinking that usually result in damages during process of feeding or retraction of the laser fiber. The present disclosure also allows gripping of the laser fiber during feeding and retraction which prevents the laser fiber from, thereby preventing any hygiene issues that might arise through contamination. The present disclosure allows a constant amount of pressure to be applied on the laser fiber resulting in reduced risks of laser fiber breakages during the process of feeding and retracting of the laser fiber. The present disclosure further reduces number of assisting personnel during the dusting and has a systematic and hassle free process of handling the laser fiber during the dusting.

The ureteroscope in the present disclosure is a generic device that may be applied in different flexible ureteroscopes which are used in places where flexible ureteroscopy is performed. The present disclosure of providing an automated motor assisted movement of the laser fiber during feeding and retraction may also be applied to various flexible scopes with a working channel within which a laser fiber or other devices of similar dimensions are required to be fed or retracted. Such flexible scopes are used for different minimally invasive surgeries, for example a laparoscopic procedure.

The terms “an embodiment”, “embodiment”, “embodiments”, “the embodiment”, “the embodiments”, “one or more embodiments”, “some embodiments”, and “one embodiment” mean “one or more (but not all) embodiments of the invention(s)” unless expressly specified otherwise.

The terms “including”, “comprising”, “having” and variations thereof mean “including but not limited to”, unless expressly specified otherwise.

The enumerated listing of items does not imply that any or all of the items are mutually exclusive, unless expressly specified otherwise.

The terms “a”, “an” and “the” mean “one or more”, unless expressly specified otherwise.

A description of an embodiment with several components in communication with each other does not imply that all such components are required. On the contrary a variety of optional components are described to illustrate the wide variety of possible embodiments of the invention.

When a single device or article is described herein, it will be readily apparent that more than one device/article (whether or not they cooperate) may be used in place of a single device/article. Similarly, where more than one device or article is described herein (whether or not they cooperate), it will be readily apparent that a single device/article may be used in place of the more than one device or article or a different number of devices/articles may be used instead of the shown number of devices or programs. The functionality and/or the features of a device may be alternatively embodied by one or more other devices which are not explicitly described as having such functionality/features. Thus, other embodiments of the invention need not include the device itself.

The illustrated operations of FIG. 5 show certain events occurring in a certain order. In alternative embodiments, certain operations may be performed in a different order, modified or removed. Moreover, steps may be added to the above described logic and still conform to the described embodiments. Further, operations described herein may occur sequentially or certain operations may be processed in parallel. Yet further, operations may be performed by a single processing unit or by distributed processing units.

Finally, the language used in the specification has been principally selected for readability and instructional purposes, and it may not have been selected to delineate or circumscribe the inventive subject matter. It is therefore intended that the scope of the invention be limited not by this detailed description, but rather by any claims that issue on an application based here on. Accordingly, the disclosure of the embodiments of the invention is intended to be illustrative, but not limiting, of the scope of the invention, which is set forth in the following claims.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

We claim:

1. A ureteroscope for dusting stones in a body cavity with a laser fiber, the ureteroscope comprising:
a flexible probe comprising a proximal end and a distal end, the flexible probe comprising:
a working channel configured to accommodate the laser fiber in the distal end of the flexible probe at a working position, the laser fiber to be deflected against a target stone for dusting; and
a handle extending proximally from the flexible probe, the handle comprising:
a plurality of control switches configured to provide digital control signals, the digital control signals configured to control a motor assisted movement of the laser fiber in the working channel by a user, the motor assisted movement of the laser fiber comprising one of a feeding movement and a retracting movement; and
a laser fiber feeder and retraction module configured to control one of the feeding movement and the retracting movement of the laser fiber in the working channel, the feeding movement of the laser fiber comprising a forward movement of the laser fiber in a first rotating direction, and the retracting movement of the laser fiber comprising a backward movement of the laser fiber in a second rotating direction.

2. The ureteroscope as claimed in claim 1, wherein the flexible probe further comprises:
a deflecting tip at the distal end of the flexible probe and configured to be deflected and in turn deflect the laser fiber, the deflecting tip being positioned at a minimum distance from the target stone;
an image viewing system configured to view the laser fiber extending from the deflecting tip and the target stone; and
a light source configured to provide light to assist viewing of the laser fiber and the target stone through the image viewing system.

3. The ureteroscope as claimed in claim 1, wherein the laser fiber feeder and retraction module comprises:
a light dependent resistor (LDR) configured to generate a digital LDR signal in response to sensing presence of the laser fiber in the working channel, the laser fiber being manually introduced into the working channel and positioned at an initial position;
a processing module coupled to the LDR and configured to generate a digital motor signal in response to the digital LDR signal and the digital control signals;
a driver coupled to the processing module and configured to generate an analog motor signal in response to the digital motor signal;
a motor coupled to the driver and configured to turn a number of steps based on the analog motor signal; and
a plurality of pulleys coupled to the motor and configured to control the motor assisted movement of the laser fiber in the working channel.

4. The ureteroscope as claimed in claim 3, wherein the processing module further comprises:
a controller comprising:
an input and output interfacing module configured to receive the digital LDR signal from the LDR;
a first plurality of input and output interfacing modules configured to receive the digital control signals from the plurality of control switches;
a decision making module configured to determine presence of the laser fiber in the working channel based on the digital LDR signal, and to determine type of the feeding movement and the retracting movement of the laser fiber based on the digital control signals; and
a second plurality of input and output interfacing modules coupled to the decision making module and configured to receive an output data signal from the decision making module and to generate the digital motor signal.

5. The ureteroscope as claimed in claim 3, wherein the initial position is a predefined distance of the laser fiber in the working channel prior to the feeding movement of the laser fiber by the laser fiber feeder and retraction module.

6. The ureteroscope as claimed in claim 1, wherein the working position is a predefined distance of the laser fiber from the distal end of the flexible probe and is defined by laser energy settings and composition of the target stone.

7. The ureteroscope as claimed in claim 1, wherein the laser fiber is energized to a power level and a frequency level to enable the dusting of the target stone.

8. A method of dusting stones in a body cavity with a laser fiber in a ureteroscope, the method comprising:
receiving, by a processing module of a laser fiber feeder and retraction module of the ureteroscope, a digital light dependent resistor signal from a light dependent resistor (LDR) and digital control signals from a plurality of control switches, the digital LDR signal being generated in response to sensing presence of the laser fiber in a working channel of a flexible probe of the ureteroscope, the laser fiber being manually introduced into the working channel and positioned at an initial position and configured to be advanced to a working position, the digital control signals configured to control a motor assisted movement of the laser fiber in the working channel by a user, the motor assisted movement of the laser fiber comprising one of a feeding movement and a retracting movement;
processing, by the processing module, the digital LDR signal and the digital control signals to generate an analog motor signal; and
initiating, by the processing module, activation of a motor in the laser fiber feeding and retraction module in response to the analog motor signal to control one of the feeding movement and the retracting movement of the laser fiber in the working channel for dusting of a target stone, the feeding movement of the laser fiber comprising a forward movement of the laser fiber in a first rotating direction, and the retracting movement of the laser fiber comprising a backward movement of the laser fiber in a second rotating direction.

9. The method as claimed in claim 8, wherein the laser fiber is energized to a power level and a frequency level to enable the dusting of the target stone.

10. The method as claimed in claim 9, wherein the laser fiber extending from a deflecting tip of the flexible probe is viewed by an image viewing system in the flexible probe with assistance of light provided by a light source in the flexible probe.

11. The method as claimed in claim 10, wherein the feeding movement and the retracting movement of the laser fiber is controlled by:
generating, by the processing module, an output data signal corresponding to the digital LDR signal and the digital control signals, the output data signal defining the presence of the laser fiber in the working channel based on the digital LDR signal and type of the feeding movement and the retracting movement of the laser fiber;
processing, by the processing module, the output data signal to generate the analog motor signal; and
initiating, by the processing module, driving of the motor to turn a number of steps based on the analog motor signal to move the laser fiber in one of the feeding movement and the retracting movement of the laser fiber using a plurality of pulleys.

12. The method as claimed in claim 11, and further comprising:
determining, by the processing module, the type of the feeding movement and the retracting movement of the laser fiber based on the digital control signals, the type of feeding movement of the laser fiber comprising one of a fine feed and a coarse feed of the laser fiber, the type of retracting movement of the laser fiber comprising one of a fine retract and a coarse retract of the laser fiber.

13. The method as claimed in claim 8, wherein the initial position is a predefined distance of the laser fiber in the working channel prior to the feeding movement of the laser fiber by the laser fiber feeder and retraction module.

14. The method as claimed in claim 8, wherein the working position is a predefined distance of the laser fiber from a distal end of the flexible probe and is defined by laser energy settings and composition of the target stone.
